(19) United States
(12) Patent Application Publication
(10) Pub. No.: US 2010/0273204 A1
(43) Pub. Date: Oct. 28, 2010

(54) METHODS FOR MONITORING THE EFFICACY OF ANTI-IL-2R ANTIBODIES IN MULTIPLE SCLEROSIS PATIENTS

(75) Inventor: James Peter Sheridan, III, Mountain View, CA (US)

Correspondence Address: Facet Biotech Corporation, ATTN; Legal Department, 1400 Seaport Blvd., Redwood City, CA, 94063, US

(73) Assignee: FACET BIOTECH CORPORATION, Redwood City, CA (US)

(21) Appl. No.: 12/768,131

(22) Filed: Apr. 27, 2010

Related U.S. Application Data
(60) Provisional application No. 61/173,138, filed on Apr. 27, 2009.

(51) Int. Cl. C12Q 1/02

(52) U.S. Cl. 435/29

(57) ABSTRACT

The use of HLA-DR⁺CD4⁺ T cell counts as biomarker for the efficacy of daclizumab treatment in patients diagnosed with multiple sclerosis.

### 1. CROSS REFERENCE TO RELATED APPLICATIONS

This application claims benefit under 35 U.S.C. §119(e) to application Ser. No. 61/173,138, filed on Apr. 27, 2009, the entire contents of which are incorporated herein by reference.

### 2. STATEMENT REGARDING FEDERALLY SPONSORED RESEARCH

Not applicable.

### 3. BACKGROUND

The goal of multiple sclerosis (MS) treatment is to prevent permanent disabilities and delay disease progression. A shorter-term therapeutic goal focuses on reducing the relapse rate. IFN-beta is the most commonly used chronic maintenance agent for treating MS. Drawbacks to IFN-beta treatment include inadequate response in at least 30% of patients following initiation of IFN-beta therapy (Bertolotto, et al., 2002, J Neurosurg Psychiatry, 73(2):148-153; and Durelli, 2004, J Neurol., 251 Suppl 4:IV13-1V24), and induction of anti-interferon antibodies in 7% to 42% of patients (Sorensen, 2003, Lancet, 362:1184-91). Although other agents are used to treat MS, including corticosteroids, glatiramer acetate, mitoxantrone, and natalizumab, these agents are only partially effective in managing clinical relapses, and some carry significant safety risks.

Because the agents currently being used to treat MS are not completely effective in managing the disease, it is desirable to identify and clinically validate markers that can be used to monitor the clinical response in an individual diagnosed with MS to a therapeutic agent as means for optimizing and/or changing the treatment regime for that individual.

### 4. SUMMARY

One agent that has shown promise for treating MS in clinical trials is daclizumab. Daclizumab was evaluated as a treatment for MS in a Phase 2, randomized, double-blinded, placebo-controlled, multi-center, dose-ranging study (the CHOICE study). At the end of the 24-week dosing period, compared to IFN-beta placebo, there was a 25% reduction in new or enlarged gadolinium contrast enhancing lesions (Gd-CEL) as detected by magnetic resonance imaging (MRI) in the daclizumab 1 mg/kg group and a 72% reduction in the daclizumab 2 mg/kg group (FIG. 1). Both daclizumab regimes were associated with an approximate 35% reduction in annualized relapse rate at 24 weeks (Montalban, X. et al., Multiple Sclerosis, 13: S18-S18 Suppl. 2 Oct. 2007; and, Kaufman, M. D., et. al., Neurology, 70 (11): A220-A220 Suppl. 1 Mar. 11, 2008).

Patient blood samples were collected throughout the CHOICE study and analyzed to determine levels of immune cell subsets and associated activation markers prior to, during, and after treatment with daclizumab. Rapid, reversible, reductions in HLA-DR⁺ CD4⁺ T cells, a subset of T cells, were observed during the daclizumab treatment phase for the 1 mg/kg and 2 mg/kg dosing groups. The exposure-dependent reductions in activated T cell numbers were consistent with the exposure-dependent reductions in new or enlarged Gd-CEL and reductions of annualized relapse rate.

These results indicate that changes in HLA-DR⁺CD4⁺ T cell counts can be used as a biomarker of clinical response to daclizumab in a patient diagnosed with MS. Accordingly, the methods described herein disclose the use of HLA-DR⁺CD4⁺ T cell counts to monitor the efficacy of daclizumab in a patient diagnosed with MS. In some embodiments, the method comprises determining the level of HLA-DR⁺CD4⁺ T cells in a patient diagnosed with MS following exposure to daclizumab, wherein a decrease in the level of HLA-DR⁺CD4⁺ T cells indicates that daclizumab is effective in ameliorating at least one symptom of MS in the treated patient. Biological samples from the treated patient can be collected at one or more times prior to, during, and/or after treatment with daclizumab.

Symptoms of MS that can be stabilized or improved using the methods described herein include, but are not limited to, reducing the relapse rate, stabilizing or reducing the rate of disability progression as measured by standard scores such as the Expanded Disability Status Scale (EDSS) score, decreasing the number of new or enlarged Gd-CEL, and/or decreasing the number of new or enlarged T2 MRI lesions. The subject being treated can have relapsing forms of MS, including relapsing/remitting MS, secondary progressive MS, progressive relapsing MS, or worsening relapsing MS. In addition to daclizumab, other IL-2R antibodies, such as monoclonal antibodies, chimeric antibodies, humanized antibodies, or fully human antibodies that specifically bind to the alpha or p55 (Tac) chain of the IL-2 receptor can be used in the methods described herein.

It is to be understood that both the foregoing general description and the following detailed description are exemplary and explanatory only and are not restrictive of the compositions and methods described herein. In this application, the use of the singular includes the plural unless specifically state otherwise. Also, the use of “or” means “and/or” unless state otherwise. Similarly, “comprise,” “comprises,” “comprising,” “include,” “includes” and “including” are not intended to be limiting.

### 5. BRIEF DESCRIPTION OF THE FIGURES

FIG. 1 is a bar graph depicting data demonstrating the reduction in gadolinium contrast enhancing lesions at the end of a 24 week dosing period with daclizumab;

FIG. 2 is a line graph depicting data demonstrating rapid, reversible, reductions in HLA-DR⁺CD4⁺ activated T cells during daclizumab treatment phase; and

FIG. 3 is a line graph depicting data demonstrating the relationship between daclizumab steady state area under the curve (AUC) and decreases in HLA-DR⁺CD4⁺ activated T cell counts wherein a reduction in HLA-DR⁺CD4⁺ cells is represented as a positive number on the y-axis.

### 6. DETAILED DESCRIPTION

### 6.1 Definitions

As used herein, the following terms are intended to have the following meanings:

The term “antibody” refers to an immunoglobulin molecule that specifically binds to, or is immunologically reactive with, a particular antigen, and includes polyclonal, monoclonal, genetically engineered (e.g., rIgG) and otherwise modified forms of antibodies, including but not limited to chimeric antibodies, humanized antibodies, heteroconjugate antibodies (including, e.g., bispecific antibodies), antigen binding fragments of antibodies, including e.g., Fab′, F(ab′)₂, Fab, Fv, and scFv fragments and multimerici forms of antigen binding fragments, including e.g., diabodies, triabodies and tetrabodies. Moreover, unless otherwise indicated, the term “monoclonal antibody” (mAb) is meant to include both intact molecules, as well as, antibody fragments (such as, for example, Fab and F(ab′)₂ fragments) which are capable of specifically binding to a protein. Fab and F(ab′)₂ fragments lack the Fc fragment of intact antibody, clear more rapidly from the circulation of the animal or plant, and may have less non-specific tissue binding than an intact antibody (Wahl et al., 1983, J. Nucl. Med. 24:316).

The term “scFv” refers to a single chain Fv antibody in which the variable domains of the heavy chain and the light chain from a traditional antibody have been joined to form one chain.

References to “VH” refer to the variable region of an immunoglobulin heavy chain of an antibody, including the heavy chain of an Fv, scFv, or Fab. References to “VL” refer to the variable region of an immunoglobulin light chain, including the light chain of an Fv, scFv, dsFv or Fab. Antibodies (Abs) and immunoglobulins (Igs) are glycoproteins having the same structural characteristics. While antibodies exhibit binding specificity to a specific target, immunoglobulins include both antibodies and other antibody-like molecules which lack target specificity. Native antibodies and immunoglobulins are usually heterotetrameric glycoproteins of about 150,000 daltons, composed of two identical light (L) chains and two identical heavy (H) chains. Each heavy chain has at one end a variable domain (VH) followed by a number of constant domains. Each light chain has a variable domain at one end (VL) and a constant domain at its other end.

Complementarity determining regions (CDRs) are also known as hypervariable regions both in the light chain and the heavy chain variable domains. The more highly conserved portions of variable domains are called the framework (FR). As is known in the art, the amino acid position/boundary delineating a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The disclosure provides antibodies comprising modifications in these hybrid hypervariable positions. The variable domains of native heavy and light chains each comprise four FR regions, largely by adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the target binding site of antibodies (See Kabat et al., Sequences of Proteins of Immunological Interest (National Institute of Health, Bethesda, Md. 1987). As used herein, numbering of immunoglobulin amino acid residues is done according to the immunoglobulin amino acid residue numbering system of Kabat et al., unless otherwise indicated.

The term “antibody fragment” refers to a portion of a full-length antibody, generally the target binding or variable region. Examples of antibody fragments include Fab, Fab′, F(ab′)2 and Fv fragments. An “Fv” fragment is the minimum antibody fragment which contains a complete target recognition and binding site. This region consists of a dimer of one heavy and one light chain variable domain in a tight, non-covalent association (VH-VL dimer). It is in this configuration that the three CDRs of each variable domain interact to define a target binding site on the surface of the VH-VL dimer. Collectively, the six CDRs confer target binding specificity to the antibody. However, even a single variable domain (or half of an Fv comprising only three CDRs specific for a target) has the ability to recognize and bind target, although at a lower affinity than the entire binding site. “Single-chain Fv” or “scFv” antibody fragments comprise the VH and VL domains of an antibody, wherein these domains are present in a single polypeptide chain. Generally, the Fv polypeptide further comprises a polypeptide linker between the VH and VL domains which enables the scFv to form the desired structure for target binding.

The Fab fragment contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′ fragments are produced by cleavage of the disulfide bond at the hinge cysteines of the F(ab′)₂ pepsin digestion product. Additional chemical couplings of antibody fragments are known to those of ordinary skill in the art.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

The determination of whether two antibodies bind substantially to the same epitope is accomplished using methods known in the art, such as a competition assay. In conducting an antibody competition study between a control antibody (for example, daclizumab) and any test antibody, one may first label the control antibody with a detectable label, such as, biotin, an enzyme, radioactive label, or fluorescent label to enable the subsequent identification. In such an assay, the intensity of bound label is measured in a sample containing the labeled control antibody and the intensity of bound label sample containing the labeled control antibody and the unlabeled test antibody is measured. If the unlabeled test antibody competes with the labeled antibody by binding to an overlapping epitope, the detected label intensity will be decreased relative to the binding in the sample containing only the labeled control antibody. Other methods of determining binding are known in the art.

The term “monoclonal antibody” refers to an antibody that is derived from a single clone, including any eukaryotic, prokaryotic, or phage clone, and not the method by which it is produced. Antibodies immunologically reactive with a particular antigen can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow and Lane, “Antibodies: A Laboratory Manual,” Cold Spring Harbor Laboratory Press, New York (1988); Hammerling et al., in: “Monoclonal Antibodies and T-Cell Hybridomas,” Elsevier, N.Y. (1981), pp. 563 681 (both of which are incorporated herein by reference in their entireties).

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Any of the anti-IL-2R antibodies described herein can be chimeric.

The term “humanized antibody” or “humanized immunoglobulin” refers to an immunoglobulin comprising a human framework, at least one and preferably all complementarity determining regions (CDRs) from a non-human antibody, and in which any constant region present is substantially identical to a human immunoglobulin constant region, i.e., at least about 85%, at least 90%, and at least 95% identical. Hence, all parts of a humanized immunoglobulin, except possibly the CDRs, are substantially identical to corresponding parts of one or more native human immunoglobulin sequences. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos. 5,530,101; 5,585,089; 5,693,761; 5,693,762; 6,180,370 (each of which is incorporated by reference in its entirety). Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489 498 (1991); Studnicka et al., Prot. Eng. 7:805 814 (1994); Roguska et al., Proc. Natl. Acad. Sci. USA, 91:969 973 (1994), and chain shuffling (U.S. Pat. No. 5,565,332), all of which are hereby incorporated by reference in their entireties. The anti-IL-2R antibodies described herein include humanized antibodies, such as mouse humanized antibodies, fully human antibodies, and mouse antibodies.

An “anti-IL-2R antibody” is an antibody that specifically binds an IL-2 receptor. For example, in some embodiments, an anti-IL-2R antibody binds the high affinity IL-2 receptor (K_{d}˜10 pM). This receptor is a membrane receptor complex consisting of the two subunits: IL-2R-alpha (also known as T cell activation (Tac) antigen, CD25, or p55) and IL-2R-beta (also known as p75 or CD122). In other embodiments, an anti-IL-2R antibody binds the intermediate affinity IL-2 receptor (K_{d}=100 pM), which consists of the p75 subunit and a gamma chain. In other embodiments, an anti-IL-2R antibody binds the low affinity receptor (K_{d}=10 nM), which is formed by p55 alone.

Anti-IL-2R antibodies suitable for use in the methods described herein include monoclonal antibodies, chimeric antibodies, humanized antibodies, or fully human antibodies. Examples of anti-IL-2R antibodies capable of binding Tac (p55) include, but are not limited to, Zenapax®, the chimeric antibody basiliximab (Simulect®), BT563 (see Baan et al., Transplant. Proc. 33:224-2246, 2001), and 7G8, and HuMax-TAC being developed by Genmab. The mik-beta1 antibody specifically binds the beta chain of human IL-2R. Additional antibodies that specifically bind the IL-2 receptor are known in the art. For example, see U.S. Pat. No. 5,011,684; U.S. Pat. No. 5,152,980; U.S. Pat. No. 5,336,489; U.S. Pat. No. 5,510,105; U.S. Pat. No. 5,571,507; U.S. Pat. No. 5,587,162; U.S. Pat. No. 5,607,675; U.S. Pat. No. 5,674,494; U.S. Pat. No. 5,916,559.

### 6.2 Detailed Description

MS is an inflammatory/demyelinating disease of the CNS that is one of the leading causes of neurological disability in young adults (Bielekova, B. and Martin, R., 1999, Curr Treat Options Neurol. 1:201-219). The pathogenesis observed in MS patients is, at least in part, attributable to aberrant T-cell activation. Daclizumab is a humanized antibody that binds the IL-2R alpha chain (also known as T cell activation (TAC) antigen, CD25 or p55). CD25 is present at low levels in resting human T cells, but is significantly up-regulated on activated T cells, enabling them to receive a high-affinity IL-2 signal (Waldmann, et al., 1998, Int Rev Immunol, 16:205-226). It has been proposed that selective binding of CD25 by daclizumab inhibits IL-2R signal transduction events (Goebel, J., et al., 2000, Transplant Immunol, 8:153-159, Tkaczuk, J., 2001, Transplant Proc, 33:212-213) and blocks T cell activation (Queen, C., et al., 1989, Proc Natl Acad Sci USA, 86:10029-10033).

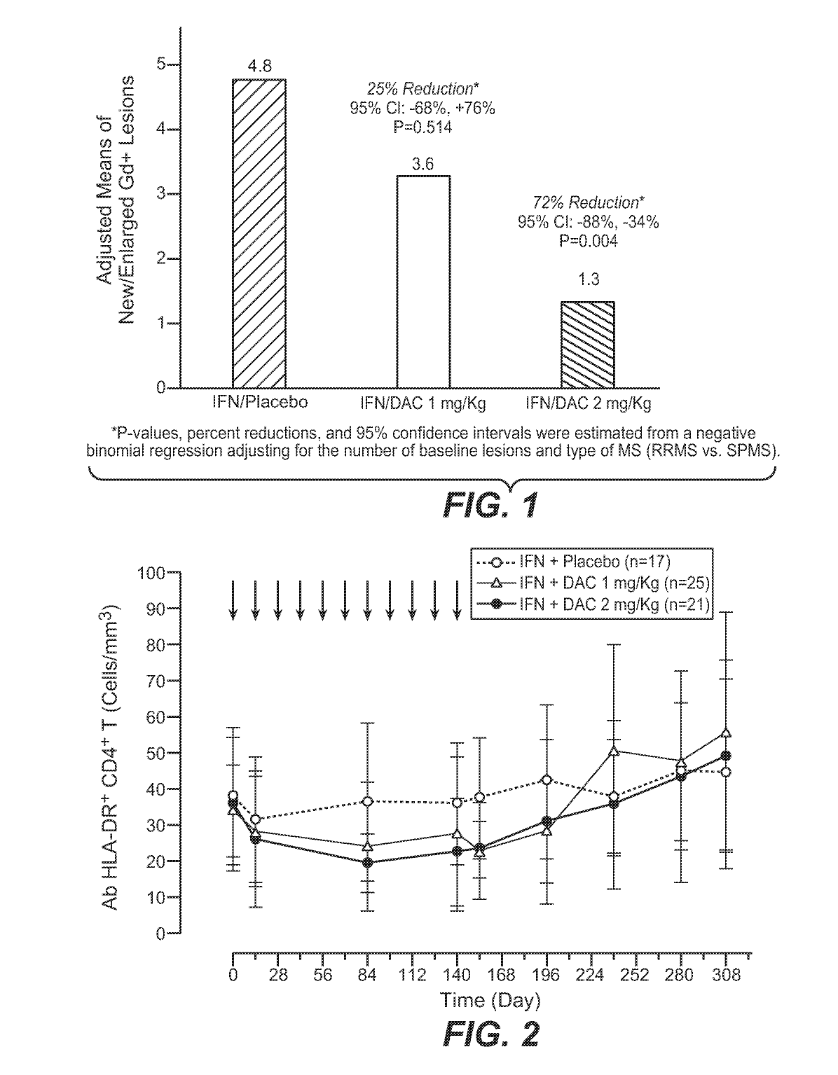

The methods described herein are based upon the discovery that rapid reductions in HLA-DR⁺CD4⁺ activated T cells are observed in MS patients treated with daclizumab (see FIG. 2). Rapid reductions in activated T cell numbers is consistent with reductions in Gd-CEL observed in daclizumab treated MS patients (Montalban, X. et al., Multiple Sclerosis, 13: S18-S18 Suppl. 2 Oct. 2007; and, Kaufman, M. D., et al., Neurology, 70 (11): A220-A220 Suppl. 1 Mar. 11, 2008). These observations suggest that changes in the level of HLA-DR⁺CD4⁺ activated T cells would be a useful tool for monitoring the clinical response in MS patients to daclizumab or an anti-IL-2R antibody capable of inhibiting IL-2R signal transduction events.

A number of studies have investigated HLA-DR⁺ expression in blood samples obtained from patients with autoimmune diseases, including MS, to determine if HLA-DR⁺ expression could be used to monitor disease progression during drug therapy. None of these studies have demonstrated that HLA-DR⁺ expression could be used to monitor disease progression in response to drug therapy. For example, Ferrarini et al., monitored the expression of HLA-DR and CD25 on T cells, as well as circulating CD3⁺ HLA-DR⁺ and CD4⁺ CD25⁺ cells in relapsing-remitting multiple sclerosis patients during interferon beta 1b therapy and determined that the expression of HLA-DR could not be used to monitor MS activity during IFN beta 1b therapy (Ferrarini et al., 1998, Multiple Sclerosis, 4:174-177). Other studies have also tried to establish a link between the expression of T-cell activation markers including HLA-DR and disease activity and/or drug therapy in MS (see, for example, Tang, M. T., et al., 2008, Multiple Sclerosis, 14:S181-S181 Suppl. 1; Aristimuno, C., et al., 2008, Journal Neuroimmunology, 204:131-135; Heinrich, A., et al., 2006, Acta Neurol Scand, 113:248-255; Sanchez-Ramon, S., et al., 2005, Immunology Letters, 96:195-201; Gelati, M., et al., 1999, J Neurol, 246:569-573; Genc, K., et al., 1997, J Clin Invest, 1:2664-2671; and Bever, C., et al., 1991, Int J Immunopharmac, 13:613-618).

Bielekova et al., reported that in MS patients treated with daclizumab there was a gradual decline in circulating CD4⁺ and CD8⁺ T cells and significant expansion of CD56^{bright} natural killer (NK) cells in vivo and this effect correlated highly with the response to daclizumab (Bielekova, B., et al., 2006, Proc Natl Acad Sci USA, 103:5941-5946). The positive correlations between expansion of CD56^{bright} NK cells and contraction of CD4⁺ and CD8⁺ T cell numbers was proposed to support the existence of an immunoregulatory pathway wherein activated CD56^{bright} NK cells inhibit T cell survival (Bielekova, B., et al., 2006, Proc Natl Acad Sci USA, 103:5941-5946).

Although Bielekova et. al. (Bielekova, B., et al., 2006, Proc Natl Acad Sci USA, 103:5941-5946) reported statistically significant reductions from baseline in total CD4+ T cell counts (mean=−11.2%, p=0.009) when MS patients (n=22) were treated with daclizumab in combination with IFN beta therapy, the observation of reduced levels of CD4⁺ T cell counts associated with daclizumab treatment were not reproduced during the placebo-controlled, blinded, CHOICE study. During the CHOICE study CD4⁺ T cell counts were obtained as often as 10 time points for each subject over the course of the 44 week study, from approximately 65 subjects, and statistically significant percent changes from baseline (parametric analysis within treatment groups) (p<0.05) were limited to a reduction (mean=18.2%) in the placebo treatment group at Week 2 (n=16), and a reduction (mean=19.1%) in the high dose daclizumab treatment group at Week 12 (n=19); there was no significant change for the low dose daclizumab treatment group.

As shown in Table 1, the observed reductions in CD4⁺ T cell counts were incidental and showed no treatment related trend. The P values shown in Table 1 were obtained from an ANOVA comparison to IFN-beta+Placebo group absolute cell counts at Week 22 of the treatment phase, and thus changes from baseline levels in CD4⁺ T cell counts observed in the two daclizumab treatment groups of the CHOICE study were not statistically significant in comparison to placebo group and were deemed to be incidental. Shown in parenthesis (n) is the number of patients sampled within each treatment group.

TABLE 1
Daclizumab associated changes in CD4⁺ T cell counts

| | | Baseline | Week 22 | Week 22 | Week 22 |
|---|---|---|---|---|---|
| Immune | | cells/mm³ | cells/mm³ | % change | P value |
| Subset | Treatment | median | median | median | (n) |
| CD4⁺ | IFN-beta + | 735 | 606 | −5.2 | (13) |
| T cells | Placebo | | | | |
| | IFN -beta + | 789 | 697 | −16.8 | 0.74 (18) |
| | DAC 1 mg/kg | | | | |
| | IFN -beta + | 652 | 546 | −20.9 | 0.82 (19) |
| | DAC 2 mg/kg | | | | |
| CD4⁺HLA- | IFN -beta + | 31.2 | 32.2 | −7.3 | (13) |
| DR⁺ | Placebo | | | | |
| | IFN -beta + | 31.1 | 20.2 | −45.7 | 0.006 (18) |
| | DAC 1 mg/kg | | | | |
| | IFN -beta + | 27.6 | 20.3 | −32.7 | 0.009 (17) |
| | DAC 2 mg/kg | | | | |

Moreover, the HLA-DR⁺CD4⁺ T cell subset typically represents a small percentage of all CD4⁺ T cells present in both healthy and diseased human subjects. The range of median percentage HLA-DR⁺CD4⁺ T cells for placebo subjects at all time points monitored during the CHOICE study was 5.5% to 6.3% of all CD4+ T cells. The maximum mean reductions from baseline in HLA-DR⁺CD4⁺ T counts as determined during the CHOICE study, mean max reduction daclizumab low dose group=−38.9%, mean max reduction daclizumab high dose group=−48.4%, were far greater than the −11.2% change in total CD4⁺ T cells reported by Bielekova et. al. (Bielekova, B., et al., 2006, Proc Natl Acad Sci USA, 103:5941-5946). The magnitude of change observed in HLA-DR⁺CD4⁺ T cell counts in response to treatment with daclizumab in the CHOICE study, supports the use of HLA-DR⁺CD4⁺ T cell counts as a sensitive marker for monitoring the efficacy of anti-IL-2R antibody therapy in MS patients.

Described herein are methods for the use of HLA-DR⁺CD4⁺ T cells as a marker for anti-IL2R antibody activity in MS patients. Typically, blood samples from MS patients are analyzed for cell surface markers using flow cytometry (see, e.g., Bielekova, B., et al., 2006, Proc Natl Acad Sci USA, 103:5941-5946). For example, the number of HLA-DR⁺CD4⁺ T cells can be analyzed using commercially available antibodies that bind preferentially to the HLA-DR⁺ protein, and in combination with fluorescent activated cell sorting (FACS), the levels of HLA-DR+ expressing cells determined The percent change in the number of HLA-DR⁺CD4⁺ T cells in response to treatment can be used to monitor the efficacy of an anti-IL-2R antibody.

In some embodiments, a reduction in the number of HLA-DR⁺CD4⁺ T cells is observed in response to treatment with an IL-2R antibody. The number of HLA-DR⁺CD4⁺ T cells can be determined by absolute cell count, i.e., the number of cells per mm³ or mm², or as a percent of total CD4+ T cells. Depending on the individual patient and the relapsing form of MS, the overall reduction in the number of HLA-DR⁺CD4⁺ T cells can vary from 25% to 100%. For example in some embodiments, the number of HLA-DR⁺CD4⁺ T cells can be reduced by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, or by at least 100%.

In some embodiments, additional cell surface markers can be monitored to provide additional information regarding the clinical response in MS patients treated with an anti-IL-2R antibody. Additional cell surface markers include, but are not limited to, CD3, CD56^{bright} NK cells, CD4, CD25, CD16, CD122, and CD8. Assays for the determination of these markers have been described, see, e.g., Bielekova, B., et al., 2006, Proc Natl Acad Sci USA, 103:5941-5946. For example, in some embodiments, the number of HLA-DR⁺CD4⁺ T cells and the number of CD56^{bright} NK cells can be analyzed and used to monitor the efficacy of an anti-IL-2R antibody.

The determination of the number of HLA-DR⁺CD4⁺ T cells generally requires that more than one sample be taken from a patient at selected times. The determination of sampling time is not critical to the methods described herein and can be selected by a medical practitioner based, in part on the length of time a patient has been treated with an anti-IL-2R antibody. Other factors that can affect sampling time include, but are not limited to, the length of time the patient has been treated for MS, which therapy(s) the patient has received prior to treatment with an anti-1′-2R antibody, and whether the patient is showing one or more of the following symptoms: increased relapse rate, an increase in the Expanded Disability Status Scale (EDSS) score, an increased number of new or enlarged Gd-CEL, and an increase in new or enlarged T2 MRI lesions. See, e.g., Perini et al., 2004, J Neurology, 251:305-309; Sorensen, et al., 2003, Lancet, 362:184-191; Pachner, 2003, Neurology, 61(Suppl 5):S2-S5; Perini et al., 2004, J Neurology, 251:305-309; and Farrell, et al., 2008, Multiple Sclerosis, 14:212-218.

For example, in some embodiments, changes in the number of HLA-DR⁺CD4⁺ T cells can be monitored before, during and after initiation of treatment with an anti-IL-2R antibody.

By way of another example, changes in the number of HLA-DR⁺CD4⁺ T cells can be monitored prior to the initiation of treatment with an anti-IL-2R antibody and at selected intervals thereafter (e.g., weekly, monthly, once every two months, once every three months, once every six months).

By way of another example, changes in the number of HLA-DR⁺CD4⁺ T cells can be monitored following initiation of treatment (e.g., within a week, within a month, within two months, within three months, within six months) with an anti-IL-2R antibody and at selected intervals thereafter (e.g., weekly, monthly, once every two months, once every three months, once every six months).

Typically, a 25% or greater reduction in the number of HLA-DR⁺CD4⁺ T cells is consistent with the administration of a therapeutically effective dose of an anti-IL-2R antibody. As used herein, a “therapeutically effective dose” is a dose sufficient to prevent advancement, decrease the relapse rate, or reduce one or more of the symptoms associated with disease progression in multiple sclerosis.

For example, in some embodiments administration of a therapeutically effective dose of an anti-IL-2R antibody to a MS patient decreases the number of relapses by at least one that occur in a given time period, such as 1 year, in the treated patient. Relapses are typically assessed by history and physical examination defined as the appearance of a new symptom or worsening of an old symptom attributable to multiple sclerosis, accompanied by an appropriate new neurological abnormality or focal neurological dysfunction lasting at least 24 hours in the absence of fever, and preceded by stability or improvement for at least 30 days (see, e.g., Sorensen, et al., 2003, Lancet, 362: 1184-1191.

In other embodiments, administration of a therapeutically effective dose of an anti-IL-2R antibody to a MS patient decreases the number of lesions detected in the patient's brain. Magnetic Resonance Imaging (MRI) of the brain is an important tool for understanding the dynamic pathology of multiple sclerosis. T₂-weighted brain MRI defines lesions with high sensitivity in multiple sclerosis and is used as a measure of disease burden. However, such high sensitivity occurs at the expense of specificity, as T₂ signal changes can reflect areas of edema, demyelination, gliosis and axonal loss. Areas of Gd-CEL demonstrated on T₁-weighted brain MRI are believed to reflect underlying blood-brain barrier disruption from active perivascular inflammation. Such areas of enhancement are transient, typically lasting <1 month. Gd-CEL brain MRI is therefore used to assess disease activity. Most T₂-weighted (T2) lesions in the central white matter of subjects with multiple sclerosis begin with a variable period of Gd-CEL. Gd-CEL and T2 lesions represent stages of a single pathological process. Brain MRI is a standard technique for assessing Gd-CEL and T2 lesions and is routinely used to assess disease progression in MS (e.g., see Lee et al., Brain 122 (Pt 7):1211-2, 1999).

As shown in FIG. 1, treatment of MS patients with daclizumab reduced the mean number of new and enlarged Gd-CEL by 25% or more. Accordingly, in some embodiments a therapeutically effective dose of an anti-IL-2R antibody to a MS patient decreases the number of Gd-CEL detected in the patient's brain by approximately 25% to 80%. In some embodiments, the number of Gd-CEL detected in the patient's brain is decreased by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%, and by at least 100%.

In other embodiments, administration of a therapeutically effective dose of an anti-IL-2R antibody to a MS patient decreases the number of T2 MRI lesions detected in the patient's brain.

In other embodiments, administration of a therapeutically effective dose of an anti-IL-2R antibody to a MS patient stabilizes a patient's disability progression as determined by the “Expanded Disability Status Scale (EDSS)” which can be used to rate neurological impairment in MS patients (Kurtzke, 1983, Neurology, 33-1444-52). The EDSS comprises 20 grades from 0 (normal) to 10 (death due to MS), progressing in a single-point step from 0 to 1 and in 0.5 point steps upward. The scores are based on a combination of functional-system scores, the patient's degree of mobility, need for walking assistance, or help in the activities of daily living. The functional-system scores measure function within individual neurological systems including visual, pyramidal, cerebellar, brainstem, sensory, bowel and bladder, cerebral and other functions.

In other embodiments, administration of a therapeutically effective dose of an anti-IL-2R antibody to an IFN-beta NAb positive MS patient reduces a patient's disability score by 10% to 75%. For example in some embodiments, a patient's disability score can be reduced by at least 10%, by at least 15%, by at least 20%, by at least 25%, by at least 30%, by at least 35%, by at least 40%, by at least 45%, by at least 50%, by at least 55%, by at least 60%, by at least 65%, by at least 70%, or by at least 75%.

In some embodiments, no change or an increase in the number of HLA-DR⁺CD4⁺ T cells following treatment of a MS patient with an anti-IL-2R antibody will be observed. In these embodiments, the patients can be assessed to determine if they are responding poorly or failing to respond to treatment with an anti-IL-2R antibody. MS patients that are responding poorly to therapy generally have a higher mean relapse rate, a higher risk of experiencing a second relapse, a higher risk of having a sustained progression of >1 on EDSS, and a lower probability of being relapse free (Malucchi, et al., 2004, Neurology, 62: 2031-2037). Accordingly, a number of clinical endpoints can be used to determine whether a patient is responding to treatment with an anti-IL-2R antibody including the frequency and rate of relapse, a 1 point or greater increase in the Expanded Disability Status Scale (EDSS) score, an increase in the number of Gd-CEL, and/or an increase in the number of T2 MRI lesions.

The data required to determine clinical endpoints can be collected at the start of the anti-IL-2R treatment and/or during follow-up visits. In some embodiments, MS patients that are responding poorly to an anti-IL-2R antibody can be treated with additional agents. For example, in some embodiments, one or more anti-IL-2R antibodies can be administered to a MS patient. In other embodiments, an anti-IL-2R antibody can be administered in combination with another MS therapy, such as an IFN-beta product. Examples of suitable IFN-beta products include, but are not limited to, one of the three IFN-beta products that have been approved: IFN-beta-1b (Betaferon®, Schering AG, Berlin, Germany), IFN-beta-1a (Avonex®, Biogen Idec, Cambridge Mass.), and IFN-beta-1a (Rebif®, Ares-Serono, Geneva, Switzerland). Non-limiting examples of other marketed drugs that may be used in combination with an anti-IL-2R treatment include glatiramer acetate (e.g., Copaxone®, Teva Pharmaceutical Industries, Ltd., Israel), corticosteroids, riluzole, azathioprine, cyclophosphamide, methotrexate, and mitoxantrone.

MS patients suitable for treatment with an anti-IL-2R antibody typically have been diagnosed with a relapsing form of multiple sclerosis including relapsing-remitting multiple sclerosis, secondary progressive multiple sclerosis, progressive relapsing multiple sclerosis and worsening relapsing multiple sclerosis. By “relapsing-remitting multiple sclerosis” herein is meant a clinical course of MS that is characterized by clearly defined, acute attacks with full or partial recovery and no disease progression between attacks. By “secondary-progressive multiple sclerosis” herein is meant a clinical course of MS that initially is relapsing-remitting, and then becomes progressive at a variable rate, possibly with an occasional relapse and minor remission. By “progressive relapsing multiple sclerosis” herein is meant a clinical course of MS that is progressive from the onset, punctuated by relapses. There is significant recovery immediately following a relapse, but between relapses there is a gradual worsening of disease progression. By “worsening relapsing multiple sclerosis” herein is meant a clinical course of MS with unpredictable relapses of symptoms, from which people do not return to normal and do not recover fully.

In some embodiments, the anti-IL-2 receptor antibody is daclizumab. The recombinant genes encoding daclizumab are a composite of human (about 90%) and murine (about 10%) antibody sequences. The donor murine anti-Tac antibody is an IgG2a monoclonal antibody that specifically binds the IL-2R Tac protein and inhibits IL-2-mediated biologic responses of lymphoid cells. The murine anti-Tac antibody was “humanized” by combining the complementarity-determining regions and other selected residues of the murine anti-TAC antibody with the framework and constant regions of the human IgG1 antibody. The humanized anti-Tac antibody daclizumab is described and its sequence is set forth in U.S. Pat. No. 5,530,101, see SEQ ID NO: 5 and SEQ ID NO: 7 for the heavy and light chain variable regions respectively. SEQ ID NOS: 5 and 7 of U.S. Pat. No. 5,530,101 are disclosed as SEQ ID NOS: 1 and 2 respectively in the sequence listing filed herewith. U.S. Pat. No. 5,530,101 and Queen et al., Proc. Natl. Acad. Sci. 86:1029-1033, 1989 are both incorporated by reference herein in their entirety.

Daclizumab has been approved by the U.S. Food and Drug Administration (FDA) for the prophylaxis of acute organ rejection in subjects receiving renal transplants, as part of an immunosuppressive regimen that includes cyclosporine and corticosteroids and is marketed by Roche as ZENAPAX®. Daclizumab also has been shown to be active in the treatment of human T cell lymphotrophic virus type 1 associated myelopathy/topical spastic paraparesis (HAM/TSP, see Lehky et al., Ann. Neuro., 44:942-947, 1998). The use of daclizumab to treat posterior uveitis has also been described (see Nussenblatt et al., Proc. Natl. Acad. Sci., 96:7462-7466, 1999).

Antibodies that bind the same (or overlapping) epitope as daclizumab can be used in the methods disclosed herein. In some embodiments, the antibody will have at least 90%, at least 95%, at least 98%, or at least 99% sequence identity with daclizumab. The antibody can be of any isotype, including but not limited to, IgG1, IgG2, IgG3 and IgG4.

In some embodiments, the antibody is basiliximab, marketed as Simulect® by Novartis Pharma AG. Basiliximab is a chimeric (murine/human) antibody, produced by recombinant DNA technology that functions as an immunosuppressive agent, specifically binding to and blocking the alpha chain of the IL-2R on the surface of activated T-lymphocytes.

Anti-IL-2R antibodies can be administered parenterally, i.e., subcutaneously, intramuscularly, intravenously, intranasally, transdermally, or by means of a needle-free injection device. The compositions for parenteral administration will commonly include a solution of an anti-IL-2R antibody in a pharmaceutically acceptable carrier. Pharmaceutically-acceptable, nontoxic carriers or diluents are defined as vehicles commonly used to formulate pharmaceutical compositions for animal or human administration. See, for example, Remington: The Science and Practice of Pharmacy, A. R. Gennaro, 20th Edition, 2001, Lippincott Williams & Wilkins, Baltimore, Md., for a description of compositions and formulations suitable for pharmaceutical delivery of the anti-IL-2R antibodies disclosed herein. See US Pat. Appl. Pub. Nos. 2003/0138417 and 2006/0029599 for a description of liquid and lyophilized formulations suitable for the pharmaceutical delivery of daclizumab.

Methods for preparing pharmaceutical compositions are known to those skilled in the art (see Remington: The Science and Practice of Pharmacy, supra). In addition, the pharmaceutical composition or formulation can include other carriers, adjuvants, or nontoxic, non-therapeutic, nonimmunogenic stabilizers and the like. Effective amounts of such diluent or carrier will be those amounts that are effective to obtain a pharmaceutically acceptable formulation in terms of solubility of components, or biological activity.

The concentration of antibody in the formulations can vary widely, i.e., from less than about 0.5%, usually at or at least about 1% to as much as 15 or 20% by weight or from 1 mg/mL to 100 mg/mL. The concentration is selected primarily based on fluid volumes, viscosities, etc., in accordance with the particular mode of administration selected.

Generally a suitable therapeutic dose of daclizumab is about 0.5 milligram per kilogram (mg/kg) to about 5 mg/kg, such as a dose of about 0.5 mg/kg, of about 1 mg/kg, about 1.5 mg/kg, about 2 mg/kg, about 2.5 mg/kg, about 3.0 mg/kg, about 3.5 mg/kg, about 4.0 mg/kg, about 4.5 mg/kg, or about 5.0 mg/kg administered intravenously or subcutaneously. Unit dosage forms are also possible, for example 50 mg, 100 mg, 150 mg, 200 mg, 300 mg, 400 mg, or up to 500 mg per dose.

Other dosages can be used to obtain serum levels of 2 to 5 μg/mL which are necessary for saturation of the Tac subunit of the IL-2 receptor to block the responses of activated T lymphocytes. Higher levels such as approximately 5 to 40 μg/mL, may be necessary for clinical efficacy. One of skill in the art will be able to construct an administration regimen to keep serum levels within the 2 to 40 μg/mL range.

Doses of basiliximab are likely to be lower, for example 0.25 mg/kg to 1 mg/kg, e.g., 0.5 mg/kg, or unit doses of 10, 20, 40, 50 or 100 mg, due to the higher affinity of basiliximab for the IL-2R target. The general principle of keeping the IL-2 receptor saturated can be used to guide the choice of dose levels of other IL-2R antibodies.

Single or multiple administrations of anti-IL-2R antibodies can be carried out with dosages and frequency of administration selected by the treating physician. Generally, multiple doses are administered. For example, multiple administration of daclizumab or other anti-IL-2R antibodies can be utilized, such as administration monthly, bimonthly, every 6 weeks, every other week, weekly or twice per week.

### 7. EXAMPLES

### Example 1

### CHOICE Study

The CHOICE study was a Phase 2, randomized, double-blinded, placebo-controlled, multi-center study of subcutaneous (SC) daclizumab added to interferon (IFN)-beta in the treatment of active, relapsing forms of MS. Results from the CHOICE study confirmed that daclizumab at 2 mg/kg every two weeks significantly decreased the number of new Gd-CEL in patients who have active, relapsing forms of MS on concurrent IFN-beta therapy (Montalban, X. et al., Multiple Sclerosis, 13: S18-S18 Suppl. 2 OCT 2007; and, Kaufman, M. D., et. al., Neurology, 70 (11): A220-A220 Suppl. 1 Mar. 11, 2008). A smaller decrease in new or enlarging Gd-CEL was observed for those study subjects receiving 1 mg/kg daclizumab every four weeks.

A patient was enrolled in the study once he or she was randomized. Enrolled patients remained on their baseline IFN-beta regimen and were randomized in a 1:1:1 ratio to one of the following 3 treatment arms (see Table 2).

TABLE 2

| | | No. Total | No. |
|---|---|---|---|
| Treatment Arm¹ | Dose Level and Frequency | Dosing Visits | Patients |
| A (High Dose)² | Daclizumab SC: | 11 | 55 |
| | 2 mg/kg q2 weeks × | | |
| | 11 doses | | |
| B (Low Dose)³ | Daclizumab SC: | 11 | 55 |
| | 1 mg/kg q4 weeks × | | |
| | 6 doses | | |
| C (Placebo)⁴ | Placebo SC: | 11 | 55 |
| | q2 weeks × 11 doses | | |

¹All patients continue on prior regimen of IFN-beta SC/IM for the duration of the study.
²Patients in Arm A (high dose) receive 2 SC injections (2 daclizumab 1 mg/kg) for 11 dosing visits. Maximum dose daclizumab per dosing visit = 200 mg.
³Patients in Arm B (low dose) receive 2 SC injections (1 daclizumab 1 mg/kg, 1 placebo) for 6 dosing visits, alternating with 2 SC injections (2 placebo) for 5 dosing visits. Maximum dose daclizumab per daclizumab dosing visit = 100 mg.
⁴Patients in Arm C (placebo) receive 2 SC injections (2 placebo) for 11 dosing visits.

The screening period was up to 3 weeks. The treatment period was designated as 24 weeks (6 months, through Day 168) in order to include 4 weeks subsequent to the last dose of blinded study drug (Dose No. 11, which occurs at Visit No. 14, Day 140). After the treatment period, patients were followed for a total of 48 weeks (12 months) and continued IFN beta therapy for at least 5 months of this period. Total maximum time on study for each patient was approximately 18 months.

Evaluations of a given patient by EDSS and Multiple Sclerosis Functional Composite, version 3 (MSFC-3) were performed by a clinician who was not involved in the patient's treatment and was designated an “evaluating clinician.” All other assessments of the patient were under the purview of the clinician in charge of the patient's treatment (treating clinician). The MSFC-3 includes quantitative tests of: (1) Leg function/ambulation—Timed 25-foot walk (T25FW); (2) Arm function·9-Hole Peg Test (9HPT), and (3) Cognition—Paced Auditory Serial Addition Test with 3-second interstimulus intervals (PASAT3) (Cutter et al., 1999, Brain, 122(Pt 5):871-882).

Preliminary eligibility for the CHOICE study was established by history, chart inspection, and routine evaluations. During the treatment and follow up period, a number of procedures and evaluations were performed on the subjects at specified days including, but not limited to, MRI, EDSS, MSFC-3, physical exams, symptom directed physical exams, hematology/serum chemistry (e.g., for determination of pharmacokinetic assessment and anti-DAC antibodies), and blood draws for pharmacodynamic assessments and IFN-beta NAbs.

Daclizumab drug substance manufactured by PDL BioPharma, Inc. (Redwood City, Calif.) for subcutaneous delivery, was supplied in single-use vials containing 100 mg of daclizumab in 1.0 mL of 40 mM sodium succinate, 100 mM sodium chloride, 0.03% polysorbate 80, pH 6.0. Placebo was supplied in single-use vials as an isotonic solution in matching vials containing 40 mM sodium succinate, 6% sucrose, 0.03% polysorbate 80, pH 6.0.

### Example 2

### Reductions in HLA-DR⁺CD4⁺ T Cells in Response to Treatment with Daclizumab

Changes in the number HLA-DR⁺CD4⁺ T cells were determined in patients that participated in the CHOICE study (see Example 1). HLA-DR⁺CD4⁺ T cell levels were analyzed in patients that received daclizumab 2 mg/kg every 2 weeks (high dose), daclizumab 1 mg/kg every 4 weeks (low dose), or placebo SC added to interferon beta therapy for 24 weeks. Patient's whole blood specimens were collected into vacutainer tubes containing anti-coagulant using standard venipuncture techniques and shipped via overnight courier for immediate, treatment-blinded, analysis using fluorescent activated cell sorting (FACS). Commercially available antibodies were obtained from BD Biosciences (USA) that bind preferentially to the HLA-DR protein (e.g., antibody clone L243(G45-6)) and that bind preferentially to specific immune subsets (e.g. antibodies that bind selectively to CD3 (e.g., clone SK7) and CD4 (e.g., clone SK3) antigens were used in combination with FACS to determine levels of HLA-DR+ expressing cells when patients were treated with DAC antibody. Specimen preparation and FACS assay procedures were performed in accordance with the practices recommended in the “Guideline for Flow Cytometric Immunophenotyping” (see Calevetti et. al., 1993, Cytometry 14:702-714). Parametric (paired t test) and non-parametric (Wilcoxon) analyses were performed comparing levels of immune subset cell counts between dosing groups. Individual daclizumab exposure characteristics (results of post hoc analysis) from a subset of subjects, including steady state trough (C_{ss,min}), and AUCₛₛ, were used, separately, as predictors to model changes from baseline level in individual immune subsets or changes over time (calculated area under the change from baseline-time curve (AUC)). Relationship between changes from baseline level of putative activated T cell subsets and total new or enlarged Gd-CEL between Weeks 8 and 24 was also evaluated using linear correlation, negative binomial correlation, ANOVA or Kruskal-Wallis Tests statistical analysis approaches.

Analysis of daclizumab treatments at a dose of 2 mg/kg SC every 2 weeks, added to background treatment with interferon-beta produced statistically and clinically meaningful reductions in MS lesion activity (see FIG. 1) when compared to remaining on interferon-beta alone (and adding placebo).

Rapid reductions in absolute activated T cells were observed among patients in both daclizumab dose groups, but not in placebo-treated patients (see FIG. 2). As shown in FIG. 2, decreases from baseline in levels of absolute HLA-DR⁺ activated CD4⁺ T cells were observed for both dosing groups. Statistically significant reductions (p<0.05) in comparison to placebo are indicated (* DAC low dose and ** DAC high dose). Absolute levels of HLA-DR+ activated CD4+ T cells returned to approximate baseline levels at the time of CD25 de-saturation between days 196 to 280. At the last sampling at the end of the treatment period (week 22), mean levels in HLA-DR⁺CD4⁺ T cells were significantly lower in both DAC dose groups compared with placebo (DAC low dose vs. placebo: 22.5 vs. 37.4 cells/mcL, P=0.006; DAC high dose vs. placebo: 23.1 vs. 37.4 cells/mcL, P=0.009). Reductions in HLA-DR⁺CD4⁺ T cells reversed following discontinuation of DAC treatment.

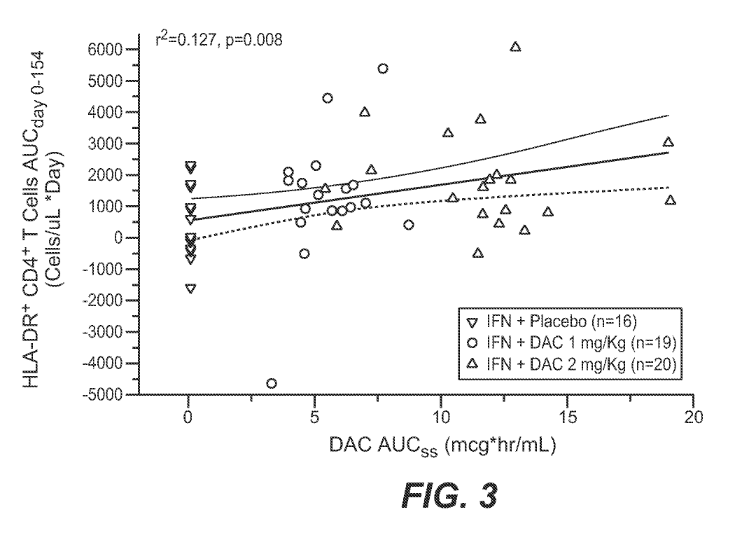

As shown in FIG. 3, a statistically significant (p=0.008) positive linear relationship was observed between individual DAC exposure at steady state and subject's reductions from baseline in levels of absolute HLA-DR⁺CD4⁺ T cell counts over time (within the dosing period), when each factor was expressed in terms of area under the curve (AUC) and when a reduction in HLA-DR⁺CD4⁺ T cell counts was expressed as a positive value (i.e. when a decrease in HLA-DR⁺CD4⁺ T cell counts from baseline was represented as a positive value). Thus, a significant positive relationship was demonstrated between daclizumab exposure and reduction in HLA-DR⁺CD4⁺ T cell counts (AUC) over time.

As shown in FIGS. 2 and 3, treatment of MS patients with daclizumab was associated with reductions in HLA-DR⁺CD4⁺ T cells; these reductions were rapid during the daclizumab treatment phase for both dosing groups and reversed after treatment with daclizumab was ended (see FIG. 2). A statistically significant, dose-dependent, positive relationship was observed between daclizumab exposure (AUCₛ) and reductions in HLA-DR⁺ CD4⁺ T cell counts (AUC) over time when a reduction in HLA-DR⁺CD4⁺ T cell counts was expressed as a positive value (i.e. when a decrease in HLA-DR⁺CD4⁺ T cell counts from baseline was represented as a positive value) (see FIG. 3). The observed exposure-dependent effect of daclizumab in reducing activated T cell numbers during the treatment phase is consistent with the exposure-dependent clinical response. Taken together, these observations support the use of monitoring HLA-DR⁺CD4⁺ T cell counts as a biomarker of daclizumab antibody treatment in MS.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method of monitoring the efficacy of an anti-IL-2R antibody in a patient diagnosed with multiple sclerosis, comprising determining the level of HLA-DR+CD4+ T cells, wherein a decrease in the level of HLA-DR+CD4+ T cells in the patient after exposure to the anti-IL-2R antibody indicates that the anti-IL-2R antibody is effective in ameliorating at least one symptom of multiple sclerosis in the treated patient.

2. A method of monitoring the efficacy of an anti-IL-2R antibody in a patient diagnosed with multiple sclerosis, comprising
(a) collecting a blood sample from said patient prior to and subsequent to administering the anti-IL-2R antibody to said patient; and
(b) determining the level of HLA-DR+CD4+ T cells in said collected blood samples, wherein a decrease in HLA-DR+CD4+ T cells subsequent to said treatment indicates that the anti-IL-2R antibody is effective in ameliorating at least one symptom of multiple sclerosis in the treated patient.

3. A method of monitoring the response to an anti-IL-2R antibody in a patient diagnosed with multiple sclerosis comprising:
(a) determining the level of HLA-DR+CD4+ T cells in a first blood sample taken from the patient prior to treatment with the anti-IL-2R antibody;
(b) determining the level of HLA-DR+CD4+ T cells in at least a second blood sample taken from the patient subsequent to the initial treatment with the anti-IL-2R antibody; and,
(c) comparing the level of HLA-DR+CD4+ T cells in the second blood sample with the level of HLA-DR+CD4+ T cells in the first blood sample; wherein a decrease in the level of HLA-DR+CD4+ T cells in the second blood sample compared to the level of HLA-DR+CD4+ T cells in the first blood sample indicates the effectiveness of the anti-IL-2R antibody treatment in ameliorating at least one symptom of multiple sclerosis in the treated patient.

4. A method of determining the efficacy of an anti-IL-2R antibody in a subject diagnosed with multiple sclerosis, comprising:
(a) obtaining a blood sample from a subject treated with an anti-Il-2R antibody;
(b) comparing the number of HLA-DR+CD4+ T cells in the blood sample to an untreated reference, wherein a decrease in the number of HLA-DR+CD4+ T cells in the blood sample compared to the untreated reference indicates that the treatment is effective for ameliorating at least one symptom of multiple sclerosis in the subject.

5. The method of claim 4, wherein the reference is a blood sample obtained from the subject prior to treatment with the anti-IL-2R antibody.

6. The method of claim 4, wherein the reference is a blood sample obtained from the subject following treatment with the anti-IL-2R antibody.

7. The method according to claim 1, 2, or 3, wherein the antibody that specifically binds the interleukin 2 receptor is a humanized antibody.

8. The method according to claim 1, 2, 3, or 4, wherein the anti-IL-2R antibody specifically binds to the alpha subunit of the human high-affinity interleukin-2 receptor and inhibits IL-2 signaling.

9. The method of claim 8, wherein the anti-IL-2R antibody is a humanized antibody.

10. The method of claim 9, wherein the humanized antibody is daclizumab.

11. The method according to claim 1, 2, 3, or 4, wherein ameliorating a symptom of multiple sclerosis comprises reducing the number of relapses in a given period.

12. The method according to claim 1, 2, or 3, wherein ameliorating a symptom of multiple sclerosis comprises reducing the rate of increase of the subject's Expanded Disability Status Score.

13. The method according to claim 1, 2, 3, or 4, wherein ameliorating a symptom of multiple sclerosis comprises reducing the number of T1 gadolinium contrast-enhanced MRI lesions.

14. The method according to claim 1, 2, 3, or 4, wherein ameliorating a symptom of multiple sclerosis comprises reducing the number of T2 MRI lesions.

15. The method of claim 10, wherein daclizumab is administered at a dose of about 0.5 to about 5 milligrams per kilogram.

16. The method of claim 10, wherein daclizumab is administered at a dose of about 1 to about 2 milligrams per kilogram.

17. The method of claim 10, wherein daclizumab is administered intravenously.

18. The method of claim 10, wherein daclizumab is administered subcutaneously, intramuscularly, intranasally, or transdermally.

19. The method of claim 10, wherein daclizumab is administered at least biweekly.

20. The method of claim 10, wherein daclizumab is administered at least monthly.

21. The method of claim 10, wherein the subject has relapsing form of multiple sclerosis.

22. The method according to claim 1, 2, 3, or 4, wherein the subject has a relapsing form of multiple sclerosis.

23. The method according to claim 21 or 22, wherein the subject has relapsing remitting, secondary progressive, progressive relapsing, or worsening relapsing multiple sclerosis.

24. A method of monitoring the efficacy of an anti-IL-2R antibody in a patient diagnosed with multiple sclerosis, comprising
(a) collecting a blood sample from said patient prior to and subsequent to administering the anti-IL-2R antibody to said patient;
(b) determining the number of HLA-DR+CD4+ T cells in said collected blood samples, to determine if a change in the number of HLA-DR+CD4+ T cells has occurred in the patient following treatment with the anti-IL-2R antibody.

25. The method according to claim 24, in which the change in the number of HLA-DR+CD4+ T cells in the treated patient is reduced by at least 25%.

26. The method according to claim 24, in which the change in the number of HLA-DR+CD4+ T cells in the treated patient is reduced by at least 50%.

27. The method according to claim 24, in which the change in the number of HLA-DR+CD4+ T cells in the treated patient is reduced by at least 100%.

28. The method according to claim 25, 26, or 27, in which the reduction in the number of HLA-DR+CD4+ T cells in the treated patient indicates that the anti-IL-2R antibody is effective in ameliorating at least one symptom of multiple sclerosis in the treated patient.

29. The method according to claim 24, wherein no change in the number of HLA-DR+CD4+ T cells in the treated patient is detected and treatment with the anti-IL-2R antibody is supplemented with one or more additional agents or terminated.

30. The method according to claim 24, wherein an increase in the number of HLA-DR+CD4+ T cells in the treated patient is detected and treatment with the anti-IL-2R antibody is supplemented with one or more additional agents or terminated.


SEQUENCE LISTING (2 sequences)

SEQ ID NO: 1 - protein, 116 aa, Artificial
  Synthetic construct.  “Variable region of the PDL humanized anti-Tac antibody heavy chain.”
QVQLVQSGAEVKKPGSSVKVSCKASGYTFTSYRMHWVRQAPGQGLEWIGYINPSTGYTEYNQKFKDKATITADESTNTAYMELSSLRSEDTAVYYCARGGGVFDYWGQGTLVTVSS

SEQ ID NO: 2 - protein, 106 aa, Artificial
  Synthetic construct.  “Variable region of the PDL humanized anti-Tac antibody light chain.”
DIQMTQSPSTLSASVGDRVTITCSASSSISYMHWYQQKPGKAPKLLIYTTSNLASGVPARFSGSGSGTEFTLTISSLQPDDFATYYCHQRSTYPLTFGQGTKVEVK